(19) United States
(12) Patent Application Publication
(10) Pub. No.: US 2004/0266805 A1
(43) Pub. Date: Dec. 30, 2004

(54) Inflammation modulatory compound

(76) Inventors: David Swift Jessop, Bristol (GB); Michael Stephen Harbuz, Bristol (GB)

Correspondence Address: Peter C Lauro, Edwards & Angell, P O Box 55874, Boston, MA, 02205, US

(21) Appl. No.: 10/488,674

(22) PCT Filed: Aug. 30, 2002
(86) PCT No.: PCT/GB02/03934

(30) Foreign Application Priority Data
Sep. 3, 2001 (GB) 0121279.4
May 3, 2002 (GB) 0210198.8

(51) Int. Cl. A61K 31/485

(52) U.S. Cl. 514/282

(57) ABSTRACT

The use of endomorphins in the treatment or prophylaxis of inflammation is described. It has been found that administration of low doses of endomorphin can prevent or reduce inflammation.

This invention relates to an inflammation modulatory compound. More particularly, the present invention relates to a naturally occurring compound which has been found to have a modulatory effect when applied to certain inflamed tissues.

Inflammation can be chronic, for example from sustained or permanent injury or from autoimmune diseases, such as systemic lupus erythematosus or rheumatoid arthritis, or be acute, for example from transient infection or from a bite such as an insect bite.

Previously, many studies have looked at the underlying mechanisms which mediate an inflammatory response and have identified various factors which are implicated as causative or corrective agents of the inflammation. The present inventors now suggest that one of these agents is the family of neuropeptides known as endomorphins. Endomorphins (EM-1 and EM-2) are hydrophobic opioid tetrapeptides with a high affinity and selectivity for the μ-opioid receptor, which is traditionally associated with pain. By virtue of this binding, many studies have investigated the role of endomorphins in pain relief (1-5).

The use of endomorphins as an anti-inflammatory agent has been suggested, for example, Khalil et al (Inflamm. Res. 1999 Oct:48 (10):550-556) (6) describes the use of high concentrations (100 μmol/l to 1 mmol/l) of endomorphin-1 by perfusion to inhibit the inflammatory response in a vacuum-induced blister model in rats (an acute inflammation model). This animal model would not be an accurate model for studies of chronic inflammation. Additionally, a problem with treatments using such high doses of endomorphins is that endomorphins have severe systemic side effects, such as suppression of respiratory function, which limits their use.

The present inventors have investigated the effect of endomorphins on chronic inflammation and have surprisingly found that much lower doses (order of magnitude lower) of endomorphins can have a modulatory effect on inflammation. “Modulatory effect” as used herein is intended to refer to the prophylaxis or prevention of inflammation from occurring and to the treatment of existing inflammation to reduce that inflammation.

Accordingly, the present invention provides the use of endomorphins in the treatment, prophylaxis or prevention of inflammation. The inflammation intended to be modulated by the present invention is primarily inflammation of voluntary or striated muscle, connective tissues, cartilage, tendons and areas surrounding or including a joint. Although all areas subject to inflammation may be modulated with the compounds of the present invention.

Preferably, the inflammation is chronic inflammation such as from an autoimmune disease. Most preferably, the inflammation is due to arthritis, especially rheumatoid arthritis. Hence, the present invention particularly provides a composition comprising endomorphin for the treatment of arthritis.

Additionally, the present invention further provides a pharmaceutically acceptable composition comprising endomorphin for use in the treatment of chronic inflammation.

The endomorphins used in the present invention may be obtained naturally or synthetically. Further, the endomorphins of the present invention may be modified, for example by glycosylation, sulphation, hydroxylation or any other known modification method for peptides, to improve their efficacy, uptake, solubility or stability either in vitro, for example in storage, or in vivo.

The endomorphins of the present invention are preferably administered by injection either locally or intra-peritoneally. However, other parenteral delivery, oral, nasal or topical application is not excluded from the scope of the present invention.

Ideally, the endomorphins of the present invention are readily incorporated into a pharmacologically acceptable preparation such as tablets, solutions, suspensions, emulsions, creams or lotions, with or without the use of micro-encapsulation technology, for oral, parenteral, injectable, inhaled, mucosal or topical delivery of the compound with the use of conventional vehicles, excipients, binders, adjuvants, preservatives or other standard pharmacological additives in common usage.

Preferably, the composition comprises an endomorphin, for example endomorphin-1 (EM-1) or endomorphin-2 (EM-2), optionally modified, or a mixture thereof, at doses of between 20 nmol and 1 fmol. More preferably, the dose range is of between 10 μmol and 20 nmol, especially for intraperitoneal injection.

In the most favoured embodiment, endomorphin, especially EM-1, is injected directly into an inflamed joint (intraplantar injection) at low doses, of between 1 fmol and 1 μmol. The present inventors have observed modulatory effects on inflammation using endomorphins within this dose range.

The preferred dose ranges may be selected according to the mode of delivery of the endomorphin. As can be seen in the Examples which follow, endomorphin can be used to treat existing inflammation or prophylactically to prevent the formation of inflammation. Very low doses of EM-1 have been found to be effective in the prevention of inflammation when administered by local injection.

Preferably, the dose of endomorphin is selected to have an anti-inflammatory effect. However, some investigators have reported that very low doses of other opioids such as beta-endorphin have a pro-inflammatory i.e. immunostimulatory effect. If this is also true for endomorphins the effect may be useful in treating diseases like HIV where it is beneficial to boost the immune system through upregulation of T-cells (7).

The present inventors have also found that the natural distribution of EM-1 and EM-2 varies according to the type of inflamed tissue. Hence, in a further aspect relating to the present invention, measurement of levels of endomorphins in tissue samples, for example biopsy or blood samples, may provide an early diagnostic marker for chronic inflammatory disease.

Embodiments of the invention will now be described by way of example only, with reference to the accompanying drawings of which

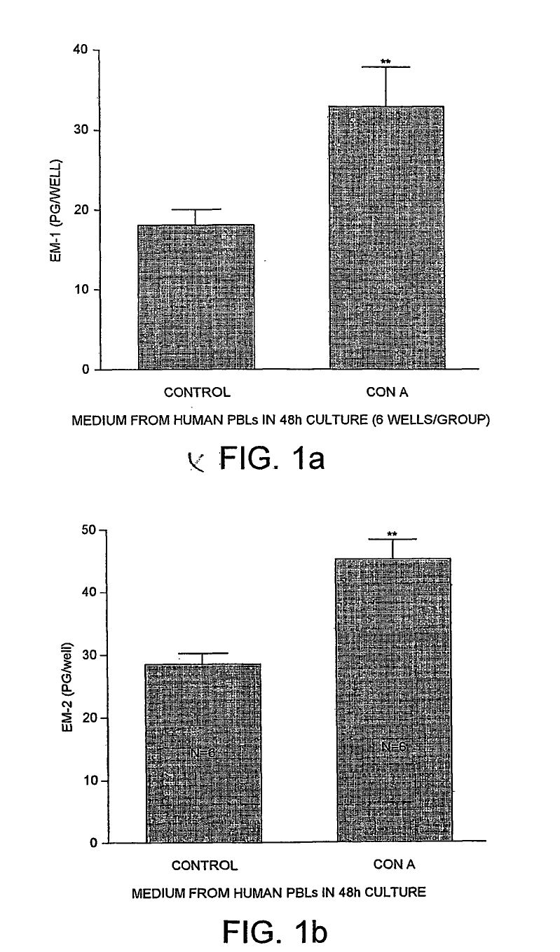

FIGS. 1A and 1B are histograms which demonstrate the secretion of EM-1 and EM-2 from cultured human lymphocytes stimulated with Concanavalin A;

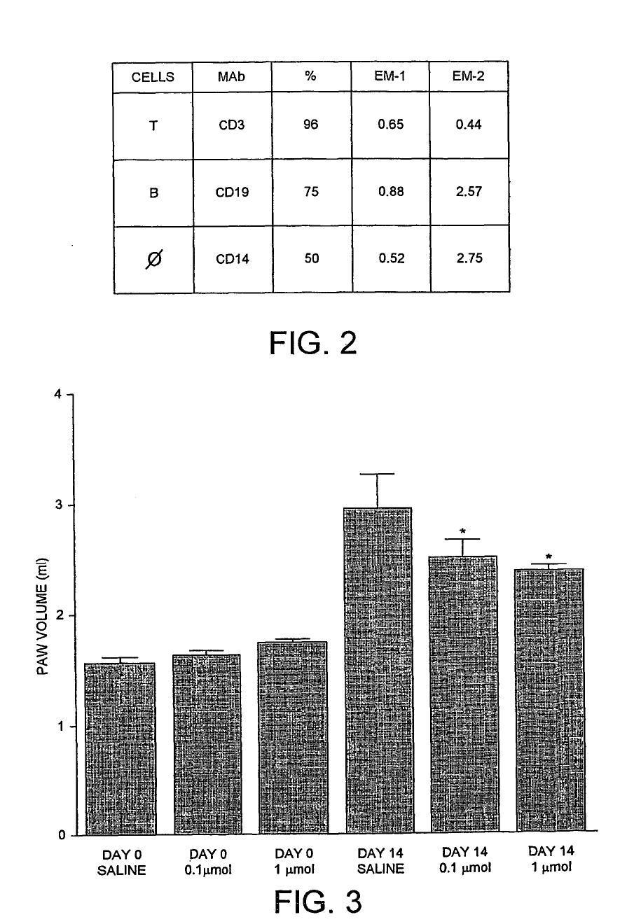

FIG. 2 is a table showing that EM-1 and EM-2 are present in enriched populations of human T-cells, B-cells and macrophages;

FIG. 3 is a histogram showing the effect of intraperitoneal injection of EM-1 on inflammation in male Wistar rats;

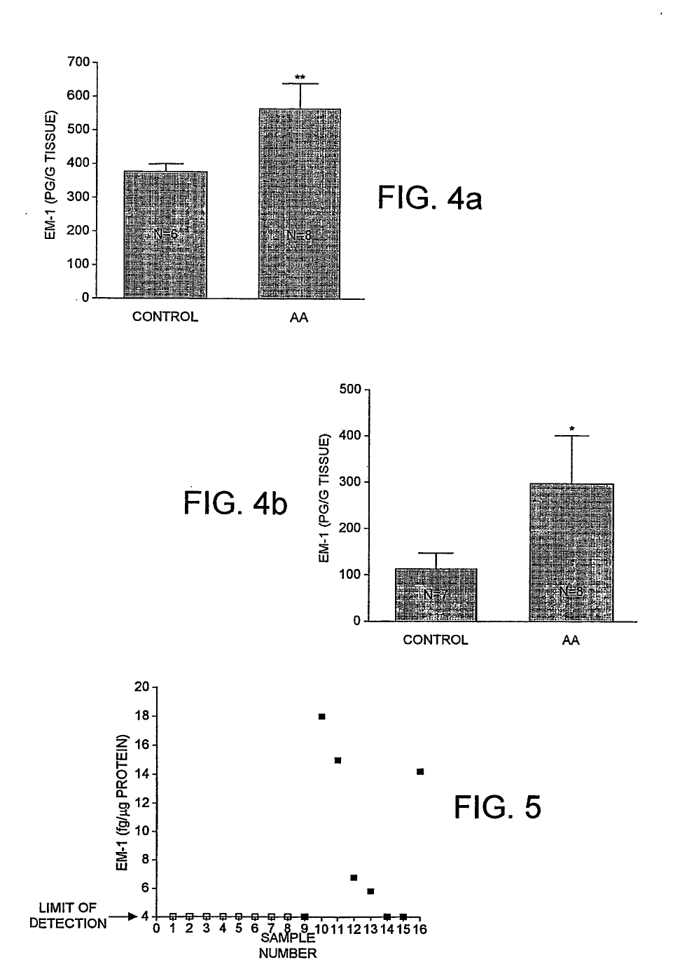

FIGS. 4a and 4b are histograms showing endomorphin-1 (EM-1) measured by radioimmunoassay in extracts of rat spleen (a) and thymus (b) from control or adjuvant arthritis (AA) rats 14 days after injection with adjuvant. Values indicated are mean±SEM; n is the group number and=6 to 8. **p<0.05; *p<0.075 M versus control by unpaired Student's t-test, and

FIG. 5 is a graph showing endomorphin-1 (EM-1) measured by radioimmunoassay in extracts of synovial tissue taken from the hind paw ankle joints of control or adjuvant arthritis (AA) rats 14 days after injection with adjuvant. Protein was measured by the bicinchoninic acid method. Radioimmunoassay limit of detection was 4 fg/μg protein. p<0.05 AA versus control by unpaired Student's t-test. □, control; ▪, AA.

### EXAMPLE 1

Localisation of EM-2 in Rat Immune Cells

A magnetic separation protocol (MACS, Miltenyi Biotech, UK) was used to enrich T cells, B cells and macrophages from adult male Wistar rat splenocyte and thymocyte populations. Cell subsets were cultured for 48 h in the presence of Con A. Cells and culture medium were acid-extracted and measured for EM-2 by radioimmunoassay (RIA) [8]. EM-2 was not detectable in any cells, but was present in culture medium from splenic (8.8 pg/million cells) and thymic macrophages (11.5 pg/million cells) and thymic B cells (7.4 pg/million cells). Splenic T and B cells secreted negligible amounts of EM-2. EM-1 was not measured. The table shown in FIG. 2 reflects data obtained by using the same protocol applied to human cells, in vitro, measuring both EM-1 and EM-2.

### EXAMPLE 2

EM-1 and EM-2 in a Rat Model of Inflammation.

Adjuvant arthritis (AA) was induced in adult male Wistar rats by a single intradermal injection (0.1 ml) of a suspension of ground, heat-killed M. butyricum in paraffin oil (10 mg/ml) into the tail base (9). Rats were despatched 14 days following injection and spleens, thymuses and hind paws were collected on dry ice and stored at −80° C. Spleens, thymuses and synovial tissue from the paws were acid-extracted and the extracts were measured for the presence of EM-1 and EM-2 by RIAs (8). EM-1 contents in the spleen (p<0.05) and thymus (p<0.075) were elevated compared to tissues from non-AA controls (FIG. 4). EM-2 contents in the spleen (3446 pg/g tissue, n=8) and thymus (1849 pg/g tissue, n=8) from AA rats were not significantly different from their respective controls (3466 pg/g tissue and 1550 pg/g tissue, n=7 per group). EM-1 was detectable in 5 out of 8 extracts of synovial tissue taken from hind paw ankle joints of AA rats exhibiting lower limb inflammation (range 5.93 to 17.94 fg/μg protein; RIA limit of detection 4 fg/μg protein), while EM-2 was detectable in 3 out of 8 extracts (8.95, 20.46 and 46.90 fg/μg protein). Neither EM-1 or EM-2 were detectable in extracts of synovial tissues from control non-AA rats (FIG. 5).

Intraperitoneal Treatment of Inflammation with Endomorphin

Adjuvant arthritis (AA) was induced in adult male Lewis rats as above. Saline vehicle or 1 μmol of endomorphin in saline was given as an intraperitoneal (ip) injection on days 11, 12 and 13 after adjuvant injection, and after the onset of inflammation, to assess the modulation effect of the endomorphin. The experiment was terminated on day 14 and the results are shown in Table I below.

TABLE I

| Compound | Paw Volume (ml) |
|---|---|
| Vehicle | 2.74 |
| Vehicle | 3.51 |
| Vehicle | 2.80 |
| Vehicle | 2.93 |
| Vehicle | 2.78 |
| Vehicle | 2.94 |
| Vehicle | 2.99 |
| Endomorphin (EM-1) | 3.15 |
| Endomorphin (EM-1) | 3.04 |
| Endomorphin (EM-1) | 2.57 |
| Endomorphin (EM-1) | 2.41 |
| Endomorphin (EM-1) | 2.57 |
| Endomorphin (EM-1) | 2.81 |
| Endomorphin (EM-1) | 2.35 |
| Endomorphin (EM-1) | 2.61 |

Vehicle n=7; mean=2.96; SEM=0.10

Endomorphin n=8; mean=2.69; SEM=0.10

Paw volume prior to inflammation was n=15; mean=2.26; SEM=0.05

One tailed t-test p=0.042 (vehicle vs EM-1 at day 14)

Hence, it can be seen that a dose of endomorphin at 1 μmol will reduce existing inflammation, and hence can be used to treat existing inflammation.

### EXAMPLE 3

Intraplantar Treatment of Inflammation With Endomorphin

Adjuvant arthritis (AA) was induced in adult male Wistar rats using the same protocol as described in Example 2 with saline vehicle or endomorphin 1 in saline being injected on days 9, 10, 11, 12 and 13 after adjuvant injection and prior to the onset of inflammation. The experiment was terminated on day 14 and the results are shown in Table II.

TABLE II

| Compound | Mean Paw Volume (ml) | Standard Deviation |
|---|---|---|
| Vehicle | 2.924 | 0.761 |
| 10 nmol EM-1^{a} | 2.260 | 0.465 |
| 1 nmol EM-1^{b} | 2.638 | 0.502 |
| 100 pmol EM-1^{c} | 2.381 | 0.417 |
| 10 pmol EM-1^{d} | 2.779 | 0.448 |
| 100 fmol EM-1^{e} | 2.450 | 0.477 |

Vehicle n=12; ^{a}n=14; ^{b}n=12; ^{c}n=14; ^{d}n=14; ^{e}n=14

Vehicle was normal saline.

ANOVA followed by Fisher PLSD test p=0.0225 (vehicle vs e) p=0.0017 (vehicle vs a) p=0.0093 (vehicle vs c).

As can be seen in Table II, a decrease in paw volume of as much as 16% is seen using doses in the femtomole range when compared to the vehicle group.

From the above data it can be seen that very low doses of endomorphins (especially endomorphin-1) can be used prophylactically to reduce or prevent the onset of inflammation.

### EXAMPLE 4

Effect of Intraperitoneal Administration of EM-1 on Inflammation

The protocol of Example 3 above was followed using intraperitoneal injection of 0.1 and 1.0 μmol Endomorphin-1 or vehicle in arthritic male Wistar rats prior to the onset of inflammation.

EM-1 or vehicle was administered on days 9, 10, 11, 12 and 13. The experiment was terminated on day 14, and the results are given in Table III below in FIG. 3.

TABLE III

| Compound | Day | Mean Paw Volume | Standard Deviation |
|---|---|---|---|
| Vehicle | 0 | 1.548 | 0.166 |
| 0.1 μmol EM1 | 0 | 1.613 | 0.141 |
| 1.0 μmol EM1 | 0 | 1.723 | 0.075 |
| Vehicle | 14 | 2.927 | 0.783 |
| 0.1 μmol EM-1 | 14 | 2.494 | 0.448 |
| 1.0 μmol EM-1 | 14 | 2.349 | 0.195 |

Vehicle n=6; 0.1 μmol n=7; 1.0 μmol n=7.

Vehicle=normal saline.

ANOVA followed by Fisher PLSD test p=<0.0001 (vehicle at day 14 vs EM-1 at day 14—both doses)

The results show that a 20% reduction in inflammation can be achieved by administration of endomorphin at 1 μmol prophylactically. The above data show that intraperitoneal administration of endomorphin can be used prophylactically to reduce or prevent the onset of inflammation.

### EXAMPLE 5

Apoptosis

Splenocytes and thymocytes obtained from adult male Wistar rats were cultured as previously described (10). Cells were incubated with EM-1, EM-2 or morphine at various concentrations for 1 h and were then activated with either Concanavalin A (ConA, 5 μg/ml, Sigma UK) or lipopolysaccharide (LPS, 25 μg/ml, Sigma, UK) and cultured for 6, 24, 48 or 72 h. After culture, cells were stained with Annexin-V-Fluos (Roche, Mannheim, Germany) and analysed by flow cytometry (Becton Dickinson, UK). Apoptosis in basal non-activated splenocytes and thymocytes was 4-8%, and pre-incubation with EM-1, EM-2 or morphine had no effect on this. ConA at 1 or 5 μg/ml induced 10-15 and 20-30% apoptosis respectively in rat splenocytes, values which were not altered by pre-incubation with EM-2 at concentrations of 10⁻⁸ to 10⁻¹⁸ mol/l. LPS at both 5 and 25 μg/ml induced 10-12% apoptosis; EM-2 did not alter this effect. Neither EM-1, EM-2 nor morphine (concentration range of morphine 10⁻⁸ to 10⁻¹⁸ mol/l) had any effect upon ConA-induced apoptosis in thymocytes. No effect of morphine, EM-1 or EM-2 was observed on apoptosis in cells following any of the culture periods.

Splenocytes and thymocytes, whether basal or ConA activated, were cultured for 6h in the presence of EM-1, EM-2 or morphine (concentration ranges as above) and were then stained with an antibody specific for rat Fas (Transduction Laboratories, Lexington, Ky., USA) and analysed by flow cytometry. No alteration was observed in expression of Fas in response to EM-1, EM-2 or morphine.

The present inventors have demonstrated that EM-1 contents in the rat spleen and thymus and in synovial tissue are increased following the onset of inflammation. This is the first published evidence that levels of endomorphin immunoreactivity are altered during a pathological condition.

The inventors measurements of EM-1 and EM-2 in immune tissues from AA rats do not permit a determination of whether EM-1 and EM-2 are synthesised in immune cells, or secreted from nerve terminals within the spleen, thymus and synovial tissue. It is possible that EM-1 and EM-2 in these tissues derive from terminals of the sympathetic nervous system. However, the present inventors have previously identified intact EM-1 and EM-2 in circulating human PBLs, (10) and have measured secretion of EM-1 and EM-2 from cultured human PBLs and rat leucocytes. These observations from rat and human studies establish unequivocally that cells of the rat and human immune system are capable of synthesising EM-1 and EM-2. Therefore the source of EM-1 and EM-2 detected in inflamed rat synovial tissues may be immune cells sequestered by the inflamed tissue.

Increased levels of EM-1 in immune and synovial tissues in AA provide further evidence for a role of immune-derived opioid peptides in modulating peripheral inflammation. Targeted delivery of β-endorphin and μ-opioid receptors to sites of inflammation (11,12) and μ-opioid-induced sensitisation of T cells to chemotaxis (13) demonstrates the existence for complex opioid regulation of inflammatory events. The mechanism of such actions however remains largely speculative. Reports that morphine could induce apoptosis in lymphocytes (14, 15) through upregulation of Fas receptors (16) suggested the possibility that endomorphins acting at morphine receptors could mediate inflammation through the Fas ligand/Fas pathway. However, using Annexin-V staining and FACS analysis the present inventors observed no effects of EM-1, EM-2, or morphine on Fas induction or apoptosis. One explanation for these discrepancies may be due to the well-recognised phenomenon that effects of compounds on immune cell functions in vitro are critically dependent upon the experimental concentrations of synthetic peptides employed and the degree of immune cell activation (17). An alternative explanation is that EM-1 is exerting immunomodulatory effects through a non-apoptotic pathway, perhaps through inhibiting the release of the pro-inflammatory peptide substance P from peripheral afferent neurons which innervate inflamed joints (18).

In Vitro Experiments on Human Cells

Expression of EM-1 and EM-2

Human peripheral blood lymphocytes (PBL's) were obtained after Ficoll-Paque purification of a buffy coat obtained from normal healthy human blood from the blood bank of Southmead Hospital, Bristol, UK. PBL's were cultured in RPMI medium (Gibco, UK) and activated by the addition of 5 μg/ml Concanavalin A (ConA). After 48 hours incubation at 37° C., the culture medium was assayed for the presence of EM-1 and EM-2 by radioimmunoassay (RIA).

Radioimmunoassays

RIAs for EM-1 and EM-2 were fully developed and validated by the present inventors. Antisera for C-terminally amidated EM-1 and EM-2 conjugated to keyhole limpet hemocyanin were raised in rabbits and supplied by Advanced Chemtech (Louisville, Ky., USA). Final antiserum titers used in the RIAs were 1:12000 and 1:300000 for EM-1 and EM-2, respectively, Synthetic EM-1 and EM-2 (Neosystem, Strasbourg, France) were iodinated with ¹²⁵I (Amersham, Herts, UK) using the chloramine T method and tracers were purified on Sep-Pak columns using a gradient of acidified 1-propanol. RIA reagents were incubated in plastic assay tubes at 4° C. for 24 h. Bound tracer was separated from unbound reagents using sheep anti-rabbit antiserum (Therapeutic Antibodies, Llandysul, Wales) in a solution of 4% polyethylene glycol. Pellets were counted for gamma radioactivity. The crossreactivity of the endomorphins EM-1 and EM-2 was investigated. Crossreactivity of EM-1 antiserum with synthetic EM-2 was 0.5% and crossreactivity of EM-2 antiserum with synthetic EM-1 was 0.01%. neither antiserum crossreacted with synthetic opioid peptides β-endorphin, dynorphin A, methionine enkephalin or orphanin FQ.

The results are shown in FIGS. 1A and 1B, where it can be seen (FIG. 1A) that approximately 70% more EM-1 is secreted from ConA stimulated cells than non-stimulated (control) cells. Similarly, it can be seen in FIG. 1B that approximately 56% more EM-2 is secreted from ConA stimulated cells than the controls.

From the above, it can be seen that in in vitro experiments increased levels of endomorphin secretion is observed from activated immune cells which may mimic events occurring at sites of inflammation.

In another experiment, purified human PBLs (obtained as above) were separated into enriched subpopulations of T-cells, B-cells and macrophages, using magnetic beads conjugated to specific human cell surface markers (Miltenyi, Germany). Subsets were extracted using acid and were measured for EM-1 and EM-2 by RIA. The results are shown in FIG. 2. EM-1 was distributed approximately evenly between the cells whereas more EM-2 was present in B-cells and macrophages than in T-cells.

Although pharmacological studies show a strong association of EM-1 and EM-2 with the μ-opioid receptor, which mediates the effects of morphine, a number of reports on central injection of endomorphins have revealed data which cannot be explained by a common receptor for endomorphins and morphine. EM-1 and EM-2 induced differential degrees of analgesia in a mouse model where similar responses were predicted (19). The present inventors were unable to block the central effects of morphine on corticosterone release by preadministration of EM-1 or EM-2 (20). EM-1 exerted analgesia without any of the immunological effects normally associated with morphine administration (21). Therefore endomorphins can exert physiological actions independent of the classical morphine receptor. At least two μ-opioid receptor subtypes exist through which EM-1 and EM-2 may act, as well as through other novel μ-opioid receptor variants. Any preferential association of endomorphins with selective μ-opioid receptor subtypes remains to be investigated. Furthermore, evidence from in vitro studies has demonstrated the existence of novel opioid receptors on immune cells with affinity for morphine and β-endorphin some orders of magnitude higher than for μ-opioid receptors on neuronal and non-activated T cells (22). Therefore the potential exists for endomorphins to exert physiological actions through novel opioid receptors at extremely low concentrations as has been reported for other opioid peptides (23,24).

In conclusion, the present inventors have identified alterations in immune and synovial tissue contents of EM-1, and to a lesser extent EM-2, in a rodent model of inflammation. Evidence that these opioids have the potential to mediate peripheral inflammatory events may lead to the development of highly specific endomorphin agonists as anti-inflammatory agents administered directly at sites of inflammation.

The inventors have also shown that intraplantar injection of EM-1 at concentrations as low as 100 fmols can reduce inflammation by as much as 16%. This is statistically significant and is likely to be beneficial by virtue of the contra-indications associated with Endomorphin treatment at high doses.

The inventors have also shown that endomorphin treatment is effective prophylactically (that is, prevention of inflammation occurring) as well as for the treatment of existing inflammation.

References

1. Stone L S, Fairbanks C A, Laughlin T M, Nguyen H O, Bushy T M, Wessendorf M W, Wilcox G L. Spinal analgesic actons of the new endogenous opioid peptides endomorphin-1 and -2. Neuroreport 1997 Sep 29;8(14):3131-3135;

2. Zadina J E, Hackler L, Ge L J, Kastin A J. A potent and selective endogenous agonist for the μ-opiate receptor. Nature 1997 Apr 3;386(6624):499-502;

3. Sakurada S, Zadina J E, Kastin A J, Katsuyama S, Fujimura T, Murayama K, Yuki M, Ueda H, Sakurada T. Differential involvement of μ-opioid receptor subtypes in endomorphin-1- and -2-induced antinociception. Eur J Pharmacol 1999 May 7;372(1):25-30

4. Przewlocka B, Mika J, Labuz D, Toth G, Przewlocki R. Spinal analgesic action of endomorphins in acute, inflammatory and neuropathic pain in rats. Eur J Pharmacol 1999 Feb 19;367(2-3):189-196;

5. Soignier R D, Vaccarino A L, Brennan A M, Kastin A J, Zadina J E. Analgesic effects of endomorphin-1 and endomorphin-2 in the formalin test in mice. Life Sci 2000 Jul 14;67(8):907-912.

6. Khalil et al. Modulation of peripheral inflammation by locally administered endomorphin-1. lnflamm. Res. 1999 Oct:48(10) 550-556.

7. Peterson P K, Gekker G, Hu S, Lokensgard J, Portoghese P S, Chao C C. Neuropharmacology 1999 Feb; 38(2):273-8 Endomorphin-1 potentiates HIV-1 expression in human brain cell cultures: implication of an atypical mu-opioid receptor.

8. Jessop, D. S., G. N. Major, T. L. Coventry, S. J. Kaye, A. J. Fulford, M. S. Harbuz & F.M. De Bree. 2000. Novel opiold peptides endomorphin-1 and endomorphin-2 are present in mammalian immune tissues. J. Neuroimmunol. 106: 53-59.

9. Harbuz, M. S., R. G. Rees, D. Eckland, D. S. Jessop, D. Brewerton & S. L. Lightman. 1992. Paradoxical responses of hypothalamic corticotropin-releasing factor (CRF) messenger ribonucleic acid (mRNA) and CRF-41 peptide and adenohypophysial proopiomelanocortin mRNA during chronic inflammatory stress. Endocrinology 130:1394-1400.

10. Richards, L. J., C. M.. Dayan, M. S. Harbuz, S. L. Lightman & D. S. Jessop. 2001. Novel opioid peptides endomorphin (EM)-1 and EM-2 are present in circulating human lymphocytes. 2002 Ann. NY. Acad. Sci, 966, 456463.

11. Mousa, S. A., Q. Zhang, N. Sifte, R. Ji R & C. Stein. 2001. beta-Endorphin-containing memory-cells and mu-opioid receptors undergo transport to peripheral inflamed tissue. J. Neuroimmunol 115:71-78.

12. Cabot, P. J., L. Carter, C. Gaiddon, Q. Zhang, M. Schafer, J. P Loeffler & C. Stein. 1997. Immune cell-derived beta-endorphin. Production, release, and control of inflammatory pain in rats. J. Clin. Invest. 100: 142-148.

13. McCarthy, L., I. Szabo, J. F. Nitsche, J. E. Pintar & T. J. Rogers. 2001. Expression of functional mu-opioid receptors during T cell development. J. Neuroimmunol. 114:173-180.

14. Fuchs, B. A., Pruett, S. B. 1993. Morphine induces apoptosis in murine thymocytes in vivo but not in vitro: involvement of both opiate and glucocorticoid receptors. J. Pharmacol. Exp. Ther. 266,417-423.

15. Fecho, K., Lysle, D. T., 2000. Heroin-induced alterations in leukocyte numbers and apoptosis in the rat spleen. Cell Immunol. 202, 113-123.

16. Yin D, Mufson R A, Wang R, Shi Y. 1999. Fas-mediated cell death promoted by opioids. Nature 397, 218.

17. Jessop, D. S., 1998. Neuropeptides: modulators of the immune system. Curr. Opin. Endocrinol. Diabetes 5, 52-58.

18. Levine, J. D., H. L. Fields & A. I. Basbaum. 1993. Peptides and the primary afferent nociceptor. J. Neurosci. 13: 2273-2286.

19. Ohsawa, M., H. Mizoguchi, M. Narita, H. Nagase, J. P. Kampine & L. F. Tseng. 2001. Differential antinociception induced by spinally administered endomorphin-1 and endomorphin-2 in the mouse. J. Pharmacol. Exp. Ther. 298:592-597.

20. Coventry, T. L., Jessop, D. S., Finn, D. P., Crabb, M. D., Kinoshita, H., Harbuz, M. S., 2001. Endomorphins and activation of the hypothalamo-pituitary-adrenal axis. J. Endocrinol. 169, 185-193.

21. Carrigan, K. A., Nelson, C. J., Lysle, D. T. 2000. Endomorphin-1 induces antinociception without immunomodulatory effects in the rat. Psychopharmacology (Berl) 151, 299-305.

22. Sharp, B. M., S. Roy & J. M. Bidlack. 1998. Evidence for opioid receptors on cells involved in host defense and the immune system. J. Neuroimmunol. 83: 45-56.

23. Kong, L. Y., M. K. McMillian, P. M. Hudson, L. Jin & J. S. Hong. 1997. Inhibition of lipopolysaccharide-induced nitric oxide and cytokine production by ultralow concentrations of dynorphins in mixed glia cultures. J. Pharmacol. Exp. Ther. 280: 61-66.

24. Williamson, S. A., R. A. Knight, S. L. Lightman & J. R. Hobbs. 1988. Effects of beta endorphin on specific immune responses in man. Immunology 65: 47-51.

1. A pharmaceutical composition comprising an endomorphin for the treatment or prophylaxis of inflammation.

2. A composition according to claim 1, in which the endomorphin is selected from the group consisting of natural endomorphins, synthetic endomorphins, endomorphin analogues, endomorphin mimetics, functional fragments of natural endomorphins, functional fragments of synthetic endomorphins and endomorphin derivatives.

3. A composition according to claim 1, in which the endomorphin is or is derived from endomorphin-1 or endomorphin-2.

4. A composition according to claim 1, in which the endomorphin is present at an effective amount to reduce or prevent inflammation.

5. A composition according to claim 1, in which the endomorphin is present at an amount to promote inflammation.

6. A composition according to claim 1, in which the endomorphin is present at a concentration of between 1 fmol and 10 pmol.

7. A composition according to 1, in which the endomorphin is present at a concentration of between 20 nmol and 10 mol.

8. A composition according to claim 1, in which the endomorphin is present at a concentration of between 1 fmol and 1 μmol in a formulation for parenteral delivery.

9. A composition according to claim 8 in which the parenteral delivery is by local or intra-peritoneal injection.

10. A composition according to claim 1, in which the composition is in the form of a tablet, solution, suspension, emulsion, cream, or lotion for oral, inhaled, mucosal or topical delivery.

11. A composition according to claim 1, in which the inflammation is chronic.

12. A composition according to claim 1 for the treatment of autoimmune disorders.

13. A composition according to claim 12 in which the autoimmune disorder is rheumatoid arthritis, systemic lupus erythematosus, multiple sclerosis, psoriasis or asthma.

14. A composition according to claim 5, in which the composition is used to upregulate T-cells production in the treatment of HIV Infection.

15. (Cancelled)
